(19) United States
(12) Patent Application Publication
(10) Pub. No.: US 2024/0425544 A1
(43) Pub. Date: Dec. 26, 2024

(54) STEREOSELECTIVE STEROIDAL REDUCTIONS

(71) Applicant: SANDHILL ONE, LLC, West Palm Beach, FL (US)

(72) Inventors: J Gregory REID, Wellington, FL (US); Jayachandra P REDDY, West Palm Beach, FL (US); SK Samad HOSSAIN, Hyberabad (IN)

(73) Assignee: SANDHILL ONE, LLC, West Palm Beach, FL (US)

(21) Appl. No.: 18/704,844

(22) PCT Filed: Nov. 1, 2022
(86) PCT No.: PCT/US2022/048624

§ 371 Date: Apr. 25, 2024

Related U.S. Application Data
(60) Provisional application No. 63/274,531, filed on Nov. 2, 2021.

(51) Int. Cl. C07J 9/00; B01J 21/18; B01J 23/44; C07J 41/00

(52) U.S. Cl. CPC C07J 9/005; B01J 21/18; B01J 23/44; C07J 41/0066

(57) ABSTRACT

Methods of making steroids, and to 5β stereoselective reductions of steroids to produce the same.

### FIELD OF THE INVENTION

The present invention relates generally to methods of making steroids, and to 5p stereoselective reductions of steroids to produce the same.

### BACKGROUND OF THE INVENTION

Cholic acid and its derivatives find utility in numerous medical applications and research initiatives. Cholic acid itself, sold under the brand name Cholbam®, is approved for use as a treatment for children and adults with bile acid synthesis disorders due to single enzyme defects, and for peroxisomal disorders (such as Zellweger syndrome). 7-Ketolithocholic acid has been examined for its effect on endogenous bile acid synthesis, biliary cholesterol saturation, and its possible role as a precursor of chenodeoxycholic acid and ursodeoxycholic acid. See Salen et al. Gasteroenterology, 1982; 83:341-7. Ursodeoxycholic acid (a/k/a UDCA or ursodiol), sold under the brand name URSO 250® and URSO Forte® tablets, is approved for the treatment of patients with primary biliary cirrhosis (PBC). More recently, obeticholic acid, sold under the brand name Ocaliva®, was approved for the treatment of PBC in combination with UDCA in adults with an inadequate response to UDCA, or as monotherapy in adults unable to tolerate UDCA.

In spite of this significant medical interest in cholic acid derivatives, methods of synthesizing the derivatives remain cumbersome and inefficient, with numerous processes being proposed. Fantin et al. Steroids, 1993 November; 58:524-526, discloses the preparation of 7α-, 12α-, 12β-hydroxy and 7α-,12α- and 7α-,12β-dihydroxy-3-ketocholanoic acids by protecting the 3-keto group as dimethyl ketal and subsequent reduction with sodium borohydride of the corresponding 7- and 12-oxo functionalities. WO 2017/079062 A1 by Galvin reports a method of preparing obeticholic acid by direct alkylation at the C-6 position of 7-keto lithocholic acid (KLCA). He et al., Steroids, 2018 December; 140:173-178, discloses a synthetic route of producing ursodeoxycholic acid (UDCA) and obeticholic acid (OCA) through multiple reactions from cheap and readily-available cholic acid. Wang et al., Steroids 157 (2020) 108600, similarly report a synthetic route of producing ursodeoxycholic acid (UDCA) through multiple reactions from commercially available bisnoralcohol (BA).

Complicating the synthetic pathway is the frequent need to reduce one or more double bonds on unsaturated intermediate compounds. Because each steroid has unique stereochemistry at several chiral centers, it would be most efficient to reduce the double bond stereo-selectively in order to obviate further chemical conversions or complex chromatographic purifications.

What is needed are more efficient processes for making cholic acid derivatives with high stereoselectivity. Particularly needed are more efficient processes for making 5β-cholic and cholanic acids, including ursodeoxycholic acid, tauroursodeoxycholic acid, and starting materials and intermediates therefor.

### SUMMARY OF INVENTION

The inventors have discovered novel methods, solvent systems, and catalytic conditions for hydrogenating the 4,5-double bond of 3-keto chol-4-enoic acids having 5-carbon side chains at the 17-position to preferentially give 5β products, that depend primarily on the use of substituted pyridines. The degree of stereoselectivity of the hydrogenation, particularly when compared to other methods using similar substrates and similar solvents, is surprisingly high and supports the commercial utility of the invention.

The methods find particular utility for reducing KCEA and related compounds, for the ultimate production of UDCA and TUDCA. Thus, in a first principal embodiment the invention provides a method of reducing a 4,5-double bond on 3-ketochol-4-enoic acid (KCEA) or a derivative thereof defined by Formula I, to preferentially give a 5f-product, comprising contacting the compound of Formula I:

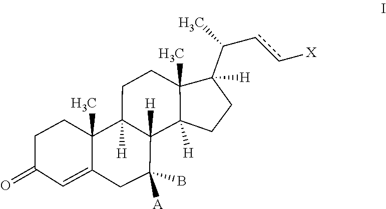

with hydrogen in the presence of a Pd catalyst in a solvent or solvent mixture comprising at least 10% of pyridine or a substituted pyridine, thereby producing the compound of Formula II:

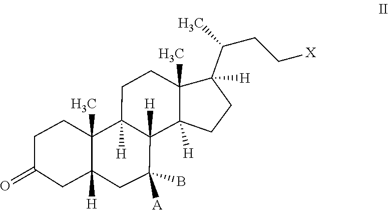

wherein: (a) A and B are OH and H respectively, H and OH respectively, H and H, or 7-oxo in combination; (b) X is C(O)OR¹ or C(O)NR¹R²; and (c) R¹ and R² are independently hydrogen, a counterion when the compound is a carboxylate or amide salt, optionally substituted C₁₋₂₀ alkyl, or optionally substituted aryl.

The methods also can be used to hydrogenate the 4,5-double bond of bisnoralcohol and related products to preferentially give 5β products. Thus, a second principal embodiment of the invention provides a method of reducing a 4,5-double bond on (20S)-21-hydroxy-20-methylpregn-4-en-3-one (BA) or a derivative thereof defined by Formula III, to preferentially give a 5β-product, comprising contacting the compound of Formula III:

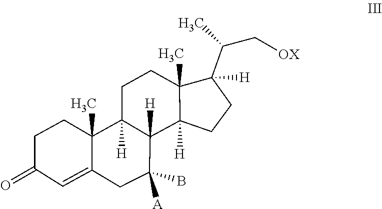

with hydrogen in the presence of a Pd catalyst in a solvent or solvent mixture comprising at least 10% pyridine or a substituted pyridine, thereby producing the compound of Formula IV:

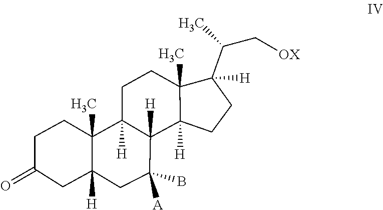

wherein: (a) A and B are OH and H respectively, H and OH respectively, H and H, or 7-oxo in combination; and (b) X is hydrogen or a protecting group.

Still further embodiments relate to the novel compounds generated by the methods of the current invention. Thus, in a third principal embodiment the invention provides a compound of Formula IV:

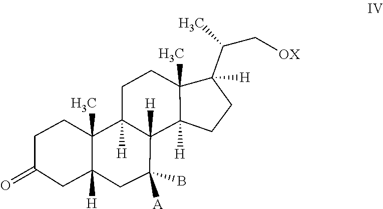

wherein: A is OX; B is H; and each X independently forms OH or a protected OH, such as an ester, an ether, a silyl ether or an acetal, or a salt thereof, with (5β,7β,20S)-7,21-dihydroxy-20-methyl-pregnan-3-one being particularly preferred.

Additional advantages of the invention are set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

### Definitions and Use of Terms

As used in the specification and claims, the singular forms a, an, and the include plural references unless the context clearly dictates otherwise. For example, the term “a specification” refers to one or more specifications for use in the presently disclosed methods and systems. “A hydrocarbon” includes mixtures of two or more such hydrocarbons, and the like. The word “or” or like terms as used herein means any one member of a particular list and also includes any combination of members of that list.

As used in this specification and in the claims which follow, the word “comprise” and variations of the word, such as “comprising” and “comprises,” means “including but not limited to,” and is not intended to exclude, for example, other additives, components, integers or steps. When an element is described as comprising one or a plurality of components, steps or conditions, it will be understood that the element can also be described as “consisting of” or “consisting essentially of” the component, step or condition, or the plurality of components, steps or conditions.

When used herein the term “about” will compensate for variability allowed for in the pharmaceutical industry and inherent in pharmaceutical products. In one embodiment the term allows for any variation within 5% of the recited specification or standard. In one embodiment the term allows for any variation within 10% of the recited specification or standard.

When a method is defined by its constituent steps, it will be understood that the method includes the steps performed consecutively, simultaneously, or in any order, unless specified to the contrary.

As used herein, the structure [symbol] refers to a bond which can be either a single covalent bond or a double bond.

In the case of Formula I or Formula III when groups A and B in combination form a 7-oxo, it is understood that the 4,5-double bond can reversibly shift to the 5,6-position and that either of the ketones may convert to its enol form to give a conjugated dienol-ketone structure.

Bisnoralcohol, (20S)-21-hydroxy-20-methylpregn-4-en-3-one, or BA, has the following chemical structure:

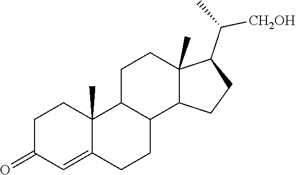

Ursodeoxycholic acid, 3α,7β-dihydroxy-5β-cholanic acid, or simply ursodiol or UDCA, has the following chemical structure:

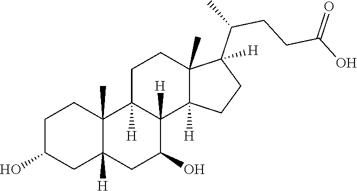

Tauroursodeoxycholic acid, or TUDCA, has the following chemical structure:

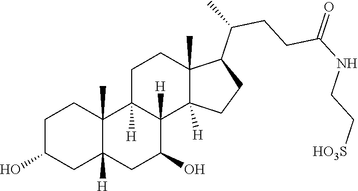

KCEA, or 3-ketochol-4-enoic acid, has the following chemical structure:

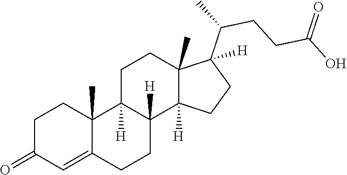

### Discussion

In a first principal embodiment the invention provides a method of reducing a 4,5-double bond on 3-ketochol-4-enoic acid (KCEA) or a derivative thereof defined by Formula I, to preferentially give a 5β-product, comprising contacting the compound of Formula I:

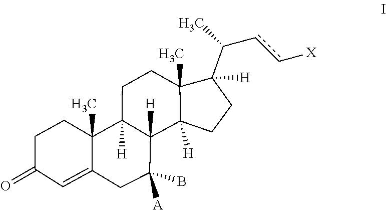

with hydrogen in the presence of a catalyst in a solvent or solvent mixture comprising at least 10% of pyridine or a substituted pyridine, thereby producing the compound of Formula II:

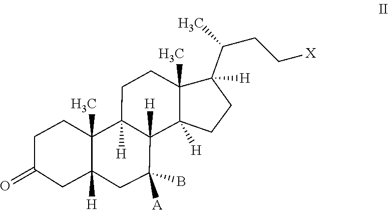

wherein: (a) A and B are OH and H respectively, H and OH respectively, H and H, or 7-oxo in combination; (b) X is C(O)OR¹ or C(O)NR¹R²; and (c) R¹ and R² are independently hydrogen, a counterion when the compound is a carboxylate or amide salt, optionally substituted C₁₋₂₀ alkyl, or optionally substituted aryl.

In one aspect of the first principal embodiment, which this document refers to as principal embodiment 1a, the invention provides a method of reducing a 4,5-double bond on a derivative of 3-ketochol-4-enoic acid (KCEA) defined by Formula I, to preferentially give a 5f-product, comprising contacting the compound of Formula I with hydrogen in the presence of a catalyst in a solvent or solvent mixture comprising at least 10% of pyridine or a substituted pyridine, thereby producing the compound of Formula II, wherein: (a) A and B are OH and H respectively, H and OH respectively, or 7-oxo in combination; (b) X is C(O)OR¹ or C(O)NR¹R²; and (c) R¹ and R² are independently hydrogen, a counterion when the compound is a carboxylate or amide salt, optionally substituted C₁₋₂₀ alkyl, or optionally substituted aryl.

In another aspect of the first principal embodiment, which this document refers to as principal embodiment 1b, the invention provides a method of reducing a 4,5-double bond on 3-ketochol-4-enoic acid (KCEA) or a derivative thereof defined by Formula I, to preferentially give a 5β-product, comprising contacting the compound of Formula I with hydrogen in the presence of a catalyst in a solvent or solvent mixture comprising at least 10% of a substituted pyridine, thereby producing the compound of Formula II, wherein: (a) A and B are OH and H respectively, H and OH respectively, H and H, or 7-oxo in combination; (b) X is C(O)OR¹ or C(O)NR¹R²; and (c) R¹ and R² are independently hydrogen, a counterion when the compound is a carboxylate or amide salt, optionally substituted C₁₋₂₀ alkyl, or optionally substituted aryl.

In another aspect of the first principal embodiment, which this document refers to as principal embodiment 1c, the invention provides a method of reducing a 4,5-double bond on 3-ketochol-4-enoic acid (KCEA) or a derivative thereof defined by Formula I, to preferentially give a 5f-product, comprising contacting the compound of Formula I with hydrogen in the presence of a catalyst in a solvent or solvent mixture comprising water and at least 10% of pyridine or a substituted pyridine, thereby producing the compound of Formula II, wherein: (a) A and B are OH and H respectively, H and OH respectively, H and H, or 7-oxo in combination; (b) X is C(O)OR¹ or C(O)NR¹R²; and (c) R¹ and R² are independently hydrogen, a counterion when the compound is a carboxylate or amide salt, optionally substituted C₁₋₂₀ alkyl, or optionally substituted aryl.

In a second principal embodiment the invention provides a method of reducing a 4,5-double bond on (20S)-21-hydroxy-20-methylpregn-4-en-3-one (BA) or a derivative thereof defined by Formula III, to preferentially give a 5β-product, comprising contacting the compound of Formula III:

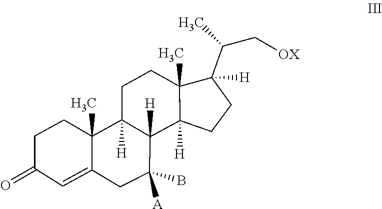

with hydrogen in the presence of a catalyst in a solvent or solvent mixture comprising at least 10% pyridine or a substituted pyridine, thereby producing the compound of Formula IV:

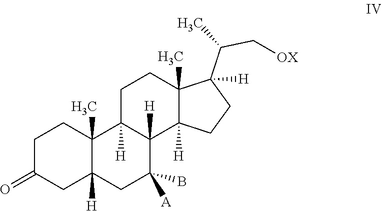

wherein: (a) A and B are OH and H respectively, H and OH respectively, H and H, or 7-oxo in combination; and (b) X is hydrogen or a protecting group.

In one aspect of the second principal embodiment, which this document refers to as principal embodiment 2a, the invention provides a method of reducing a 4,5-double bond on a derivative of (20S)-21-hydroxy-20-methylpregn-4-en-3-one (BA) defined by Formula III, to preferentially give a 5β-product, comprising contacting the compound of Formula III, with hydrogen in the presence of a catalyst in a solvent or solvent mixture comprising at least 10% pyridine or a substituted pyridine, thereby producing the compound of Formula IV, wherein: (a) A and B are OH and H respectively, H and OH respectively, or 7-oxo in combination; and (b) X is hydrogen or a protecting group.

In another aspect of the second principal embodiment, which this document refers to as principal embodiment 2b, the invention provides a method of reducing a 4,5-double bond on (20S)-21-hydroxy-20-methylpregn-4-en-3-one (BA) or a derivative thereof defined by Formula III, to preferentially give a 5β-product, comprising contacting the compound of Formula III, with hydrogen in the presence of a catalyst in a solvent or solvent mixture comprising at least 10% of a substituted pyridine, thereby producing the compound of Formula IV, wherein: (a) A and B are OH and H respectively, H and OH respectively, H and H, or 7-oxo in combination; and (b) X is hydrogen or a protecting group.

In still another aspect of the second principal embodiment, which this document refers to as principal embodiment 2c, the invention provides a method of reducing a 4,5-double bond on (20S)-21-hydroxy-20-methylpregn-4-en-3-one (BA) or a derivative thereof defined by Formula III, to preferentially give a 5β-product, comprising contacting the compound of Formula III, with hydrogen in the presence of a catalyst in a solvent or solvent mixture comprising water and at least 10% pyridine or a substituted pyridine, thereby producing the compound of Formula IV, wherein: (a) A and B are OH and H respectively, H and OH respectively, H and H, or 7-oxo in combination; and (b) X is hydrogen or a protecting group.

A third principal embodiment the invention provides a compound of Formula IV:

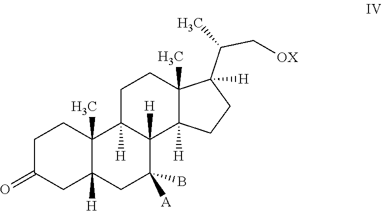

wherein: A is OX; B is H; and each X independently forms OH or a protected OH, such as an ester, an ether, a silyl ether or an acetal, or a salt thereof, with (5β,7β,20S)-7,21-dihydroxy-20-methyl-pregnan-3-one being particularly preferred.

In preferred methods of practicing the first and second principal embodiments (including principal embodiments 1a, 1b, 1c, 2a, 2b, and 2c), the Pd catalyst is a heterogeneous catalyst, i.e. in a phase different from the liquid phase in which the hydrogenation occurs. A particularly preferred catalyst is Pd on carbon, in which the Pd is supported on activated carbon in order to maximize its surface area and activity.

The solvent system is also important to the invention of the first and second principal embodiments (including principal embodiments 1a, 1b, 1c, 2a, 2b, and 2c). The hydrogenation will preferably be carried out in the presence of pyridine (except for the hydrogenation of principal embodiments 1b and 2b), or a substituted pyridine selected from 3-picoline (i.e. 3-methylpyridine), 4-picoline (i.e. 4-methylpyridine), and combinations thereof, with 3-picoline and 4-picoline being especially preferred.

The solvent system of the first and second principal embodiments (including principal embodiments 1a, 1b, 1c, 2a, 2b, and 2c) may also comprise an organic cosolvent such as dichloromethane. In various subembodiments of the first and second principal embodiments (including principal embodiments 1a, 1b, 1c, 2a, 2b, and 2c), the solvent system comprises from 10% to 90%, from 15% to 60%, or from 20% to 40% of the pyridine or substituted pyridine in combination with an organic cosolvent. The term “organic cosolvent” includes any traditional organic solvent capable of maintaining the reactants in solution, but it will be understood not to refer to pyridine or a substituted pyridine in the context of this invention, inasmuch as pyridine and substituted pyridines are separately addressed.

In various subembodiments of the first and second principal embodiments (including principal embodiments 1a, 1b, 2a, and 2b), as in principal embodiments 1c and 2c, the solvent system comprises water. In preferred embodiments the solvent system comprises from 1% to 20%, from 1.5% to 10%, or from 2% to 5% of water.

In a particularly preferred embodiment, equally applicable to methods of reducing KCEA and BA and their derivatives, and all of the principal embodiments, the solvent system comprises:
from 10% to 90% of 3-picoline or 4-picoline or a combination thereof;
from 1% to 20% water; and
the balance one or more organic solvents.

In another particularly preferred embodiment, equally applicable to methods of reducing KCEA and BA and their derivatives, and all of the principal embodiments, the solvent system comprises:
from 20% to 40% of 3-picoline or 4-picoline or a combination thereof;
from 2% to 5% water; and
the balance one or more organic solvents.

The invention can also be defined in terms of the substrate reduced in the methods of the current invention. KCEA itself is a particularly preferred substrate and forms the basis of the first principal embodiment. Other preferred substrates derived from KCEA are defined by the compound of Formula I when:
A and B are 7-oxo in combination;
A and B are H;
A is H and B is OH;
A is OH and B is H;
X is C(O)OR¹;
A and B are 7-oxo in combination and X is C(O)OR¹;
A and B are H and X is C(O)OR¹;
A is H and B is OH and X is C(O)OR¹; and
A is OH and B is H and X is C(O)OR¹.

Any of the KCEA derivatives can be further defined when [symbol] is a single bond. Alternatively, any of the KCEA derivatives can be further defined when [symbol] is a double bond. In one particular embodiment, A and B are 7-oxo in combination, X is C(O)OR¹, and [symbol] is a double bond.

Preferred KCEA substrates can also be defined by the following structures 1a, 3a, 4a, 5a, 6a, 7a, or 9a, and the resulting products defined by structures 1b, 3b, 4b, 5b, 6b, 7b, or 4b, respectively:

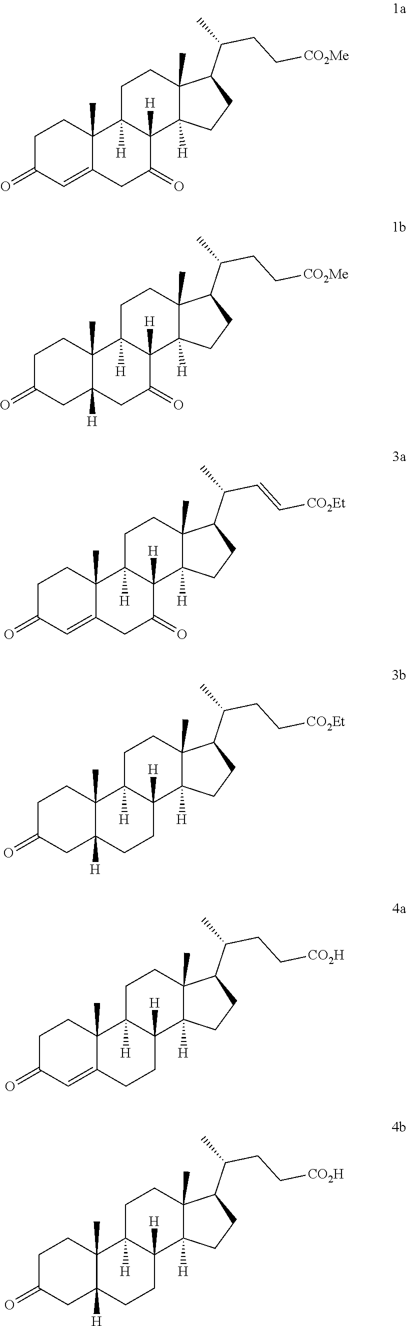

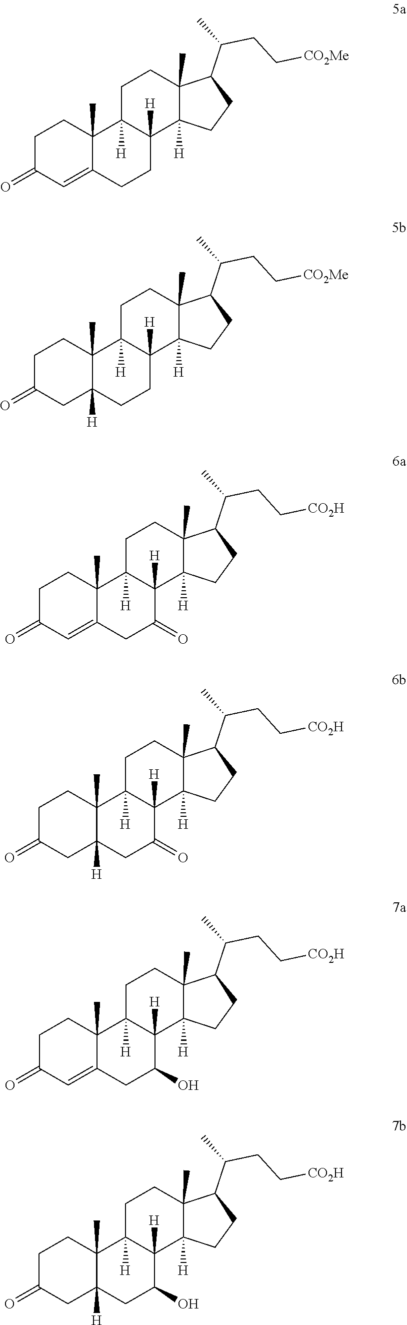

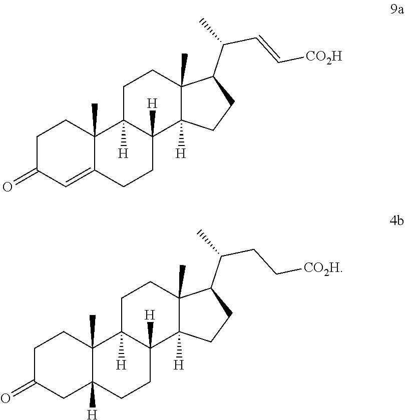

BA is also a particularly preferred substrate and forms the basis of the second principal embodiment. Other preferred substrates derived from BA are defined by the compound of Formula III when:
X forms OH or a protected OH, such as an ester, an ether, a silyl ether or an acetal;
A and B are H;
A and B are H and X forms OH or a protected OH, such as an ester, an ether, a silyl ether or an acetal;
A and B are H and X is H;
A and B are OH and H, respectively;
A and B are OH and H, respectively and X forms OH or a protected OH, such as an ester, an ether, a silyl ether or an acetal; and
A and B are OH and H, respectively, and X is H.

Preferred BA substrates can also be defined by structures 2a and 8a, with the resulting product defined by structure 2b or 8b, respectively:

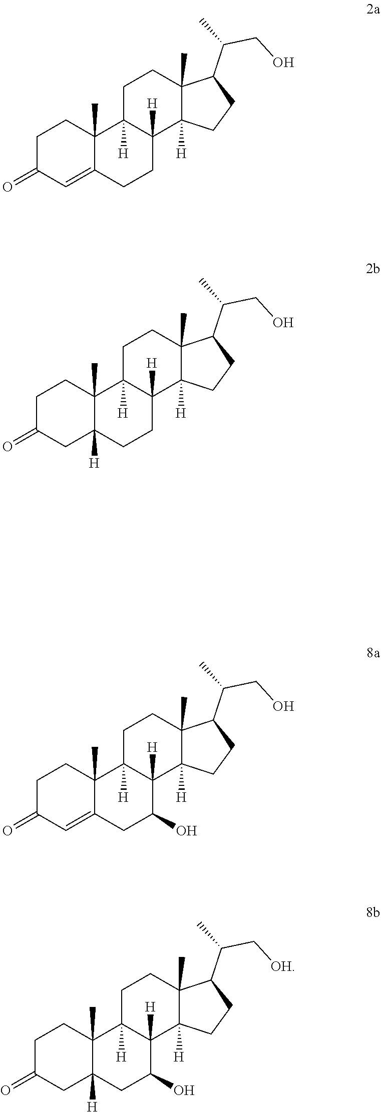

In the first principal embodiment, the methods can further be defined based on the subsequent conversion of the reduced KCEA derivative to UDCA or TUDCA, or a suitable derivative thereof.

Thus, in another subembodiment, when A and B are 7-oxo in combination the method can further comprise: (a) when X is a C(O)OR¹ ester or salt, hydrolyzing the ester or salt; (b) when X is a C(O)NR¹R² amide or salt, hydrolyzing the amide or salt to C(O)OH; (c) reducing the 3-oxo to 3α-hydroxy, and (d) reducing the 7-oxo to 7β-hydroxy, to produce UDCA. When TUDCA is desired, the method will further comprise activating the carboxyl group of UDCA and reacting with taurine to produce TUDCA.

In another subembodiment, when A and B are H, the method can further comprise: (a) when X is a C(O)OR¹ ester or salt, hydrolyzing the ester or salt; (b) when X is a C(O)NR¹R² amide or salt, hydrolyzing the amide or salt to C(O)OH; (c) reducing the 3-oxo to 3α-hydroxy, and (d) hydroxylating the 7-H to 7β-hydroxy, to produce UDCA by methods disclosed, for example, in Kollerov et al., Steroids 78 (2013) 370-378, and Sawada et al. (U.S. Pat. No. 4,579,819). When TUDCA is desired, the method will further comprise activating the carboxyl group of UDCA and reacting with taurine to produce TUDCA.

In another subembodiment, when A and B are OH and H, respectively, the method can further comprise: (a) when X is a C(O)OR¹ ester or salt, hydrolyzing the ester or salt; (b) when X is a C(O)NR¹R² amide, hydrolyzing the amide to C(O)OH; and (c) reducing the 3-oxo to 3α-hydroxy. When TUDCA is desired, the method will further comprise activating the carboxyl group of UDCA and reacting with taurine to produce TUDCA.

The second principal embodiment can also further be defined based on the subsequent conversion of the reduced BA derivative to UDCA or TUDCA, or a suitable derivative thereof. Thus, in one subembodiment, wherein A and B are H, the method further comprises: (a) converting the 21-alcohol group to a leaving group; (b) displacing the 21-leaving group with dialkylmalonate under basic conditions; (c) hydrolysis of both esters of the malonate group to give the dicarboxylic acid; (d) decarboxylation of the diacid to give the monoacid; (e) reducing the 3-oxo to 3α-hydroxy; (f) hydroxylating the 7-H to 7β-hydroxy, to produce UDCA; and (g) optionally activating the carboxyl group of UDCA and reacting with taurine to produce TUDCA.

In another subembodiment, wherein A and B are OH and H, respectively, the method further comprises: (a) selectively converting the 21-alcohol group to a leaving group; (b) displacing the 21-leaving group with dialkylmalonate under basic conditions; (c) hydrolysis of both esters of the malonate group to give the dicarboxylic acid; (d) decarboxylation of the diacid to give the monoacid; (e) reducing the 3-oxo to 3α-hydroxy, to produce UDCA; and (f) optionally activating the carboxyl group of UDCA and reacting with taurine to produce TUDCA.

### Additional Structural Definitions

A substituent is “substitutable” if it comprises at least one carbon, sulfur, oxygen or nitrogen atom that is bonded to one or more hydrogen atoms. Thus, for example, hydrogen, halogen, and cyano do not fall within this definition. If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent.

If a substituent is described as being “optionally substituted,” the substituent may be either (1) not substituted, or (2) substituted. When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. If substituents are described as being “independently selected” from a group, each substituent is selected independent of the other. Each substituent therefore may be identical to or different from the other substituent(s).

In any of the embodiments or subembodiments of this invention, a moiety which is optionally substituted may be alternatively defined as substituted with 0, 1, 2, or 3 substituents independently selected from halo, OH, amine, C₁₋₆ alkyl, C₁₋₆ alkoxy, C₁₋₆ hydroxyalkyl, CO(C₁₋₆ alkyl), CHO, CO₂H, CO₂(C₁₋₆ alkyl), and C₁₋₆ haloalkyl.

At various places in the present specification, substituents of compounds of the invention are disclosed in groups or in ranges. It is specifically intended that the invention include each and every individual sub-combination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. In any of the embodiments or subembodiments of the present invention, an alkyl group can contain from 1 to about 20, from 2 to about 20, from 1 to about 10, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons (including heteroaromatic hydrocarbons) such as, for example, phenyl, naphthyl, anthracenyl, phenanthrenyl, indanyl, indenyl, and the like. In some embodiments, aryl groups have from 6 to about 20 carbon atoms.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein “oxo” refers to ═O.

The compounds described herein can be asymmetric (e.g., having one or more stereocenters). The description of a compound without specifying its stereochemistry is intended to capture mixtures of stereoisomers as well as each of the individual stereoisomer encompassed within the genus.

The present invention also includes salts of the compounds described herein. As used herein, “salts” refers to derivatives of the disclosed compounds wherein the parent compound is modified by converting an existing acid or base moiety to its salt form. Examples of suitable salts include, but are not limited to, mineral or organic acid salts of basic residues such as amines; alkali or organic salts of acidic residues such as carboxylic acids; and the like. The salts of the present invention include the conventional non-toxic salts or the quaternary ammonium salts of the parent compound formed, for example, from non-toxic inorganic or organic acids. The salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

Finally, it will be understood that any of the novel compounds of the present invention (whether defined by a principal embodiment or a subembodiment or particular species) can be defined based on its purity and/or isolation from reaction media. Thus, in certain embodiments, the compounds are present in compositions at weight percentages greater than 10%, 50%, 90%, 95%, or 98%.

A preferred set of embodiments is defined by embodiments 1-45 below:

[Embodiment 1] A method of reducing a 4,5-double bond on 3-ketochol-4-enoic acid (KCEA) or a derivative thereof defined by Formula I, to preferentially give a 5β-product, comprising contacting the compound of Formula I:

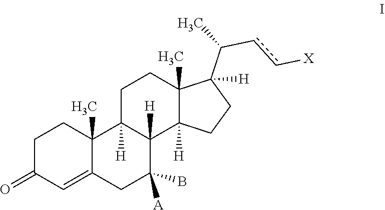

with a Pd catalyst in a solvent or solvent mixture comprising at least 10% of pyridine or a substituted pyridine, thereby producing the compound of Formula II:

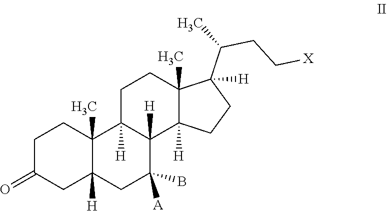

wherein:
(a) A and B are OH and H respectively, H and OH respectively, H and H, or 7-oxo in combination; (b) X is C(O)OR¹ or C(O)NR¹R²; and (c) R¹ and R² are independently hydrogen, a counterion when the compound is a carboxylate or amide salt, optionally substituted C₁₋₂₀ alkyl, or optionally substituted aryl.

[Embodiment 2] The method of Embodiment 1, wherein the Pd catalyst is a heterogeneous catalyst.

[Embodiment 3] The method of Embodiment 1, wherein the Pd catalyst is Pd on carbon.

[Embodiment 4] The method of Embodiment any of the Embodiments 1-3, wherein the solvent comprises at least 10% of 3-picoline or 4-picoline or a combination thereof.

[Embodiment 5] The method of any of Embodiments 1-3, wherein the solvent comprises an organic cosolvent in combination with from 10% to 90% of the pyridine or substituted pyridine.

[Embodiment 6] The method of any of Embodiments 1-3, wherein the solvent comprises an organic cosolvent in combination with from 10% to 90% of 3-picoline or 4-picoline or a combination thereof.

[Embodiment 7] The method of any of Embodiments 1-3, wherein the solvent comprises an organic cosolvent in combination with from 20% to 40% of the pyridine or substituted pyridine.

[Embodiment 8] The method of any of Embodiments 1-3, wherein the solvent comprises an organic cosolvent in combination with from 20% to 40% of 3-picoline or 4-picoline or a combination thereof.

[Embodiment 9] The method of any of Embodiments 1-8 wherein the solvent further comprises from 1% to 20% water.

[Embodiment 10] The method of any of Embodiments 1-8 wherein the solvent further comprises from 2% to 5% of water.

[Embodiment 11] The method of Embodiments 1-10, wherein A and B are 7-oxo in combination.

[Embodiment 12] The method of Embodiments 1-10, wherein A and B are 7-oxo in combination and X is C(O)OR¹.

[Embodiment 13] The method of any of Embodiments 1-10, wherein A and B are H.

[Embodiment 14] The method of Embodiments 1-10, wherein A and B are H and X is C(O)OR¹.

[Embodiment 15] The method of any of Embodiments 1-14, wherein [symbol] is a single bond.

[Embodiment 16] The method of any of Embodiments 1-14, wherein [symbol] is a double bond.

[Embodiment 17] The method of any of Embodiments 1-10, wherein the compound of Formula I is selected from a compound of Formula 1a, 3a, 4a, 5a, 6a, 7a, or 9a, and the compound of Formula II is selected from a compound of Formula 1b, 3b, 4b, 5b, 6b, 7b, or 4b, respectively.

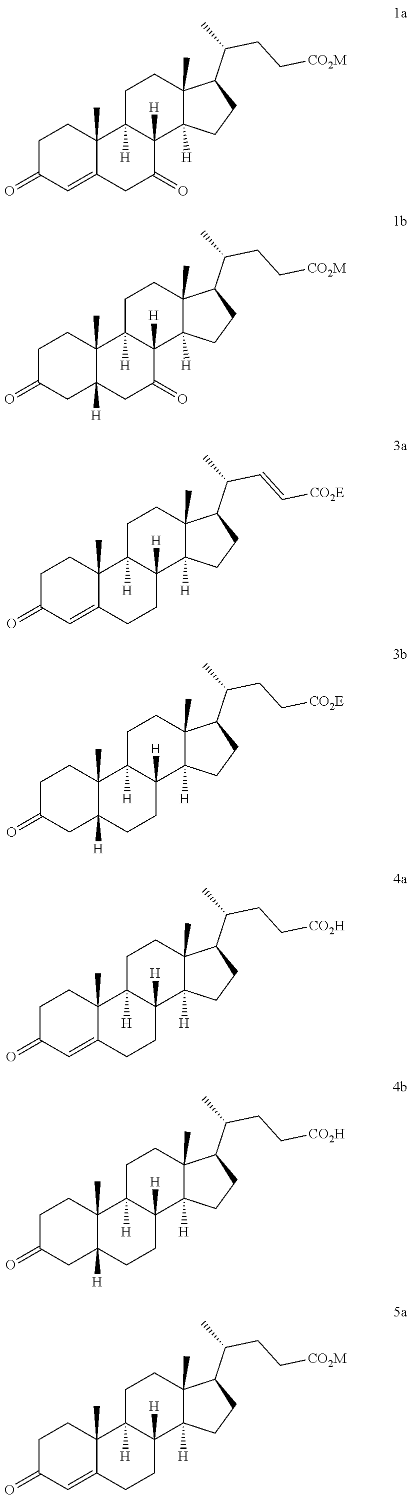

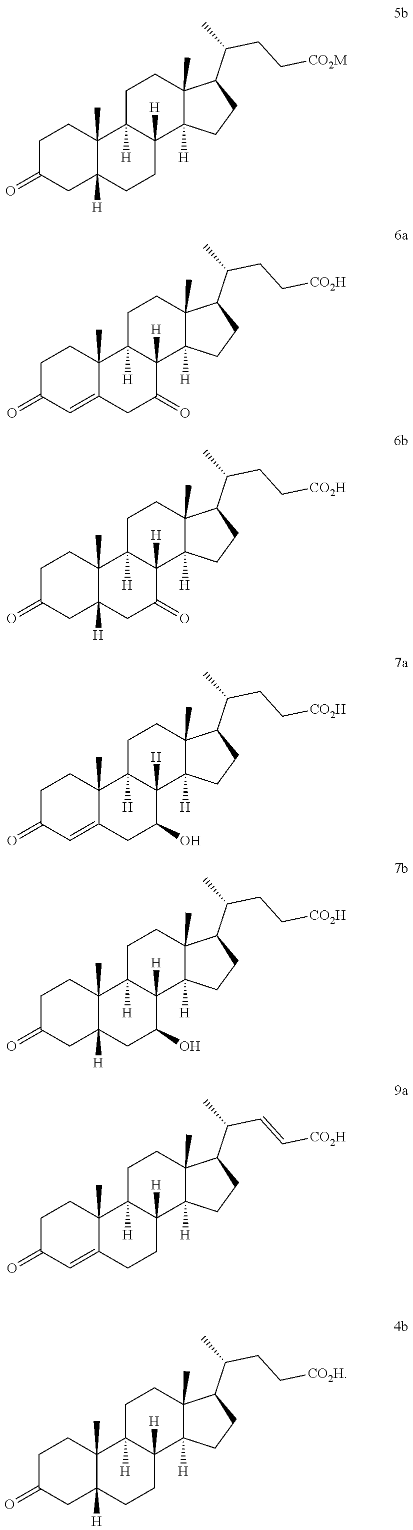

[Embodiment 18] The method of any of Embodiments 1-17, wherein A and B are 7-oxo in combination, further comprising: (a) when X is a C(O)OR¹ ester, hydrolyzing the ester; (b) when X is a C(O)NR¹R² amide, hydrolyzing the amide to C(O)OH; (c) reducing the 3-oxo to 3α-hydroxy, and (d) reducing the 7-oxo to 7β-hydroxy, to produce UDCA.

[Embodiment 19] The method of Embodiment 18, further comprising activating the carboxyl group of UDCA and reacting with taurine to produce TUDCA.

[Embodiment 20] The method of any of Embodiments 1-17, wherein A and B are H, further comprising: (a) when X is a C(O)OR¹ ester, hydrolyzing the ester; (b) when X is a C(O)NR¹R² amide, hydrolyzing the amide to C(O)OH; (c) reducing the 3-oxo to 3α-hydroxy, and (d) hydroxylating the 7-H to 7β-hydroxy, to produce UDCA.

[Embodiment 21] The method of Embodiment 20, further comprising activating the carboxyl group of UDCA and reacting with taurine to produce TUDCA.

[Embodiment 22] The method of any of Embodiments 1-17, wherein A and B are OH and H, respectively, further comprising: (a) when X is a C(O)OR¹ ester, hydrolyzing the ester; (b) when X is a C(O)NR¹R² amide, hydrolyzing the amide to C(O)OH; and (c) reducing the 3-oxo to 3α-hydroxy.

[Embodiment 23] The method of Embodiment 22, further comprising activating the carboxyl group of UDCA and reacting with taurine to produce TUDCA.

[Embodiment 24] A method of reducing a 4,5-double bond on (20S)-21-hydroxy-20-methylpregn-4-en-3-one (BA) or a derivative thereof defined by Formula III, to preferentially give a 5β-product, comprising contacting the compound of Formula III:

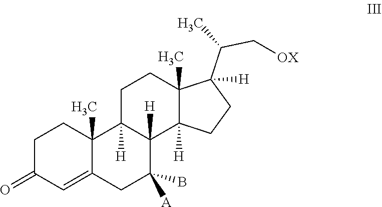

with a Pd catalyst in a solvent or solvent mixture comprising at least 10% pyridine or a substituted pyridine, thereby producing the compound of Formula IV:

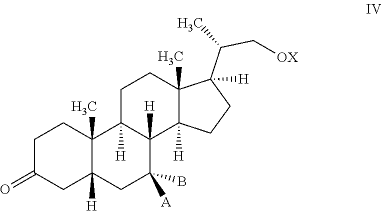

wherein: (a) A and B are OH and H respectively, H and OH respectively, H and H, or 7-oxo in combination; and (b) X is hydrogen or a protecting group.

[Embodiment 25] The method of Embodiment 24, wherein the Pd catalyst is a heterogeneous catalyst.

[Embodiment 26] The method of Embodiment 24, wherein the Pd catalyst is Pd on carbon.

[Embodiment 27] The method of any of Embodiments 24-26, wherein the solvent comprises at least 10% of 3-picoline or 4-picoline or a combination thereof.

[Embodiment 28] The method of any of Embodiments 24-26, wherein the solvent comprises an organic cosolvent in combination with from 10% to 90% of the pyridine or substituted pyridine.

[Embodiment 29] The method of any of Embodiments 24-26, wherein the solvent comprises an organic cosolvent in combination with from 10% to 90% of 3-picoline or 4-picoline or a combination thereof.

[Embodiment 30] The method of any of Embodiments 24-26, wherein the solvent comprises an organic cosolvent in combination with from 20% to 40% of the pyridine or substituted pyridine.

[Embodiment 31] The method of any of Embodiments 24-26, wherein the solvent comprises an organic cosolvent in combination with from 20% to 40% of 3-picoline or 4-picoline or a combination thereof.

[Embodiment 32] The method of any of Embodiments 24-30 wherein the solvent further comprises from 1% to 20% water.

[Embodiment 33] The method of any of Embodiments 24-31 wherein the solvent further comprises from 2% to 5% of water.

[Embodiment 34] The method of any of Embodiments 24-33, wherein X forms with the O to which it is attached an ester, an ether, a silyl ether, or an acetal.

[Embodiment 35] The method of any of Embodiments 24-33, wherein A and B are H.

[Embodiment 36] The method of any of Embodiments 24-33 wherein A and B are H and X forms with the O to which it is attached an ester, an ether, a silyl ether, or an acetal.

[Embodiment 37] The method of any of Embodiments 24-33, wherein A and B are H and X is H.

[Embodiment 38] The method of any of Embodiments 24-33, wherein A and B are OH and H, respectively.

[Embodiment 39] The method of any of Embodiments 24-33, wherein A and B are OH and H, respectively, and X forms with the O to which it is attached an ester, an ether, a silyl ether, or an acetal.

[Embodiment 40] The method of any of Embodiments 24-33, wherein A and B are OH and H, respectively, and X is H.

[Embodiment 41] The method of any of Embodiments 24-33, wherein the compound of Formula III is selected from a compound of Formula 2a or 8a and the compound of Formula IV is selected from a compound of Formula 2b or 8b, respectively:

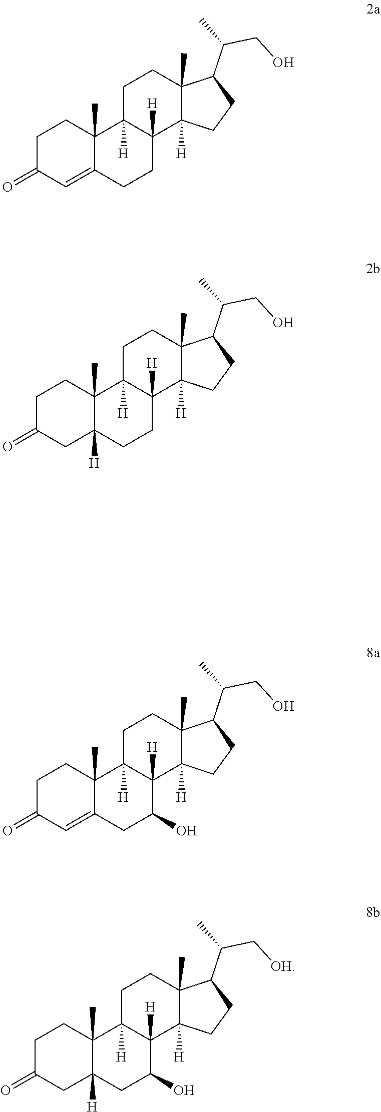

[Embodiment 42] The method of any of Embodiments 24-37 or 41, wherein A and B are H, further comprising: (a) converting the 21-alcohol group to a leaving group; (b) displacing the 21-leaving group with dialkylmalonate under basic conditions; (c) hydrolysis of both esters of the malonate group to give the dicarboxylic acid; (d) decarboxylation of the diacid to give the monoacid; (e) reducing the 3-oxo to 3α-hydroxy; (f) hydroxylating the 7-H to 7β-hydroxy, to produce UDCA; and (g) optionally activating the carboxyl group of UDCA and reacting with taurine to produce TUDCA.

[Embodiment 43] The method of any of Embodiments 24-34 or 38-41, wherein A and B are OH and H, respectively, further comprising: (a) selectively converting the 21-alcohol group to a leaving group; (b) displacing the 21-leaving group with dialkylmalonate under basic conditions; (c) hydrolysis of both esters of the malonate group to give the dicarboxylic acid; (d) decarboxylation of the diacid to give the monoacid; (e) reducing the 3-oxo to 3α-hydroxy, to produce UDCA; and (f) optionally activating the carboxyl group of UDCA and reacting with taurine to produce TUDCA.

[Embodiment 44] A compound of Formula IV:

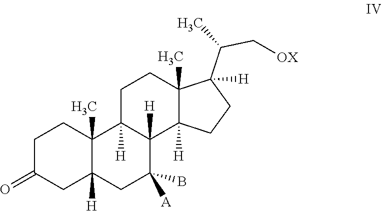

wherein: (a) A is OX; (b) B is H; and (c) each X independently forms OH or a protected OH, such as an ester, an ether, a silyl ether or an acetal, or a salt thereof.

[Embodiment 45] The compound of Embodiment 44 which is (5β,7β,20S)-7,21-dihydroxy-20-methyl-pregnan-3-one.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1. Preparation of KCEA from Bisnoralcohol

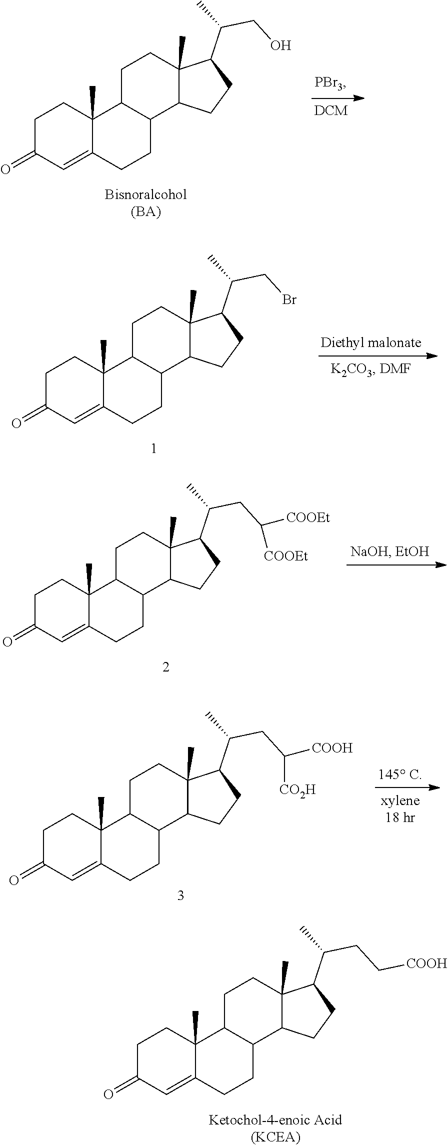

### Bromination of Bisnoralcohol:

To a stirred solution of bisnoralcohol (BA, 1 g, 3.02 mmol) in dichloromethane (DCM, 20 mL) was added PBr₃ (0.34 mL, 3.63 mmol) at 0° C. The mixture was warmed to room temperature and stirred for 3 hr, at which point TLC analysis showed complete conversion of starting material.

The reaction mixture was quenched using ice water (10 mL), stirred for 15 min and the layers were separated. The aqueous layer was extracted in DCM (10 mL) and the combined organic phase was concentrated under reduced pressure to afford compound 1 as a yellow gummy oil (crude yield 1.2 g). ¹H NMR (400 MHz, DMSO-d6): δ 5.61 (s, 1H), 3.56-3.51 (m, 1H), 3.45 (dd, J=2.1 Hz and 1.1 Hz, 1H), 2.46-2.32 (m, 2H), 2.26-2.10 (m, 2H), 199-1.89 (m, 3H), 1.82-1.70 (m, 2H), 1.70-0.82 (m, 18H), 0.70 (s, 3H) ppm.

Alkylation of Diethyl Malonate with Compound 1:

To a stirred solution of compound 1 (0.5 g, 1.27 mmol) in DMF (10 mL) was added diethyl malonate (0.58 mL, 3.812 mmol) at room temperature under N₂ atmosphere. To this solution was added K₂CO₃ (526 mg, 3.812 mmol) followed by catalytic amounts of tetrabutylammonium hydrogen sulfate (TBAHS, 43 mg, 0.127 mmol). The reaction mixture was stirred at 75-80° C. for 48 hr and TLC analysis suggested complete conversion of starting material. After completion, the reaction mixture was quenched with ice water (10 mL) and the product was extracted using ethyl acetate (2×25 mL). The combined organic layer was washed with water (20 mL) and the organic phase was concentrated under reduced pressure to obtain compound 2 as gummy oil (crude yield 700 mg). ¹H NMR (400 MHz, DMSO-d6): δ 5.61 (s, 1H), 4.0-4.20 (m, 4H), 3.50-3.42 (m, 1H), 2.43-2.30 (m, 2H), 2.27-2.10 (m, 2H), 2.11-1.90 (m, 3H), 1.89-1.70 (m, 2H), 1.62-0.80 (m, 27H), 0.63 (s, 3H) ppm. Mass analysis: m/z 473.40 [M+H]⁺ was observed.

### Hydrolysis of Compound 2:

To a stirred solution of compound 2 (12 g, 25.38 mmol) in ethanol (120 mL) was added aq. potassium hydroxide solution (7.06 g in 120 mL water, 0.127 mol) at room temperature. The reaction mixture was heated to reflux for 2 hr and TLC analysis showed complete conversion of starting material. The ethanol was evaporated under reduced pressure and the solution was diluted with water (60 mL). The mixture was washed with DCM (60 mL, to remove impurities) and the pH of the aq. layer was adjusted to ˜2 by using 6N HCl. The product was extracted using EtOAc (2×50 mL) and concentrated to dryness to afford compound 3 as a yellow solid (9.5 g).

### Decarboxylation of Compound 3:

To a 50 mL single neck round bottom flask was added compound 3 (1 g, 2.4 mmol.) in o-xylene (5 mL). The mixture was heated to reflux for 18 h and TLC analysis showed complete conversion of starting material. o-Xylene was removed under vacuum and the residue was treated with petroleum ether and the solid was filtered. The wet cake was washed with petroleum ether and dried under vacuum to afford KCEA as an off-white solid (0.5 g). ¹H NMR (400 MHz, DMSO-D6): δ 11.95 (bs, 1H), 5.62 (s, 1H), 2.44-2.34 (m, 2H), 2.28-2.06 (m, 5H), 2.0-1.91 (m, 2H), 1.87-1.74 (m, 2H), 1.72-0.81 (m, 20H), 0.69 (s, 3H) ppm.

### Example 2: Preparation of 3,7-DKCA from KCEA

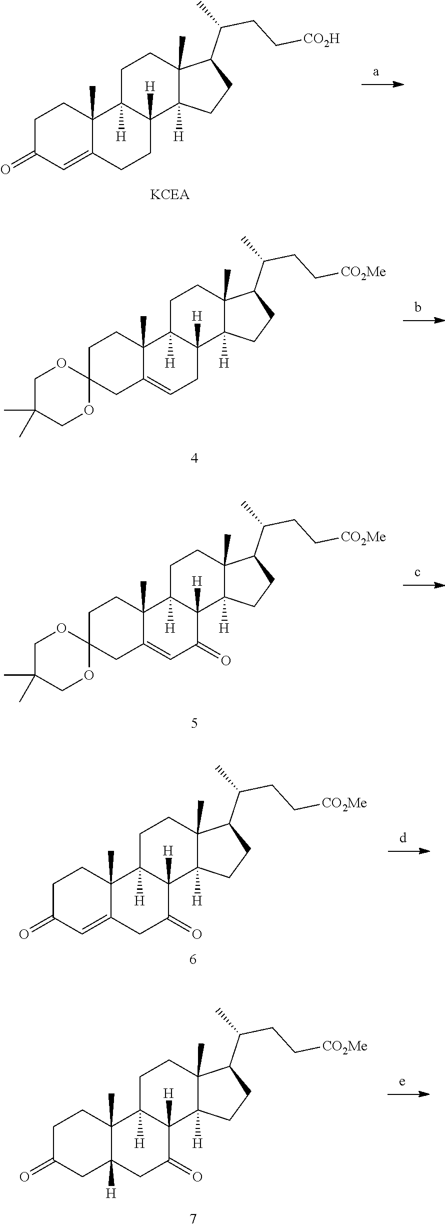

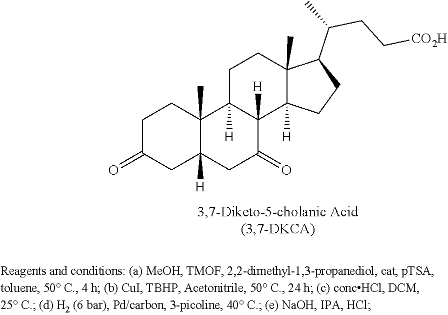

Preparation of Compound 4 from KCEA:

A 250 mL round bottom flask equipped with a stirring bar and reflux condenser was charged with toluene (90 mL), methanol (10 mL) and KCEA (10 g, 26.842 mmol). The resulting solution was inerted with nitrogen and then trimethyl orthoformate (8.8 mL, 3 equiv.) and p-toluenesulfonic acid (0.5 g, 0.1 equiv.) were added sequentially. The resulting mixture was stirred at 50-55° C. for 1 hr. The pressure was then reduced and ˜20 mL of solvent was removed via distillation. 2,2-Dimethylpropane-1,3-diol (22.3 g, 8 equiv.) and p-toluenesulfonic acid (0.5 g, 0.1 equiv.) were added and the reaction was continued for another 3 hr. At this point the mixture was cooled to 5° C. in an ice bath and treated with aqueous sodium acetate solution (30 g in 150 mL water). The mixture was stirred for 1 h at 5° C. and the resulting suspension was filtered to obtain crude product. This was purified further by silica gel chromatography to obtain Compound 4 as a white solid. (7.4 g). ¹H NMR (400 MHz, CDCl3) δ 5.38-5.33 (m, 1H), 3.68 (s, 3H), 3.60, 3.50 (ABq, 2H, J_{AB}=11.2 Hz), 3.49-3.43 (m, 2h), 2.61-0.91 (m, 37H), 0.69 (s, 3H); ESIMS for C₃₀H₄₈O₄ m/z 473.6 [M+H]⁺.

### Oxidation of Compound 4 to Compound 5:

To a solution of compound 4 (10 g) in 4:1 acetone/DCM (200 mL) at 25-35° C. is added N-hydroxyphthalimide (NHPI, 1.73 g), benzoyl peroxide (0.05 g), copper iodide (CuI, 0.04 g) and water (0.4 mL). The mixture is heated to 40-45° C. and air is bubbled through the mixture for 7 hr. The mixture is then cooled to 25-30° C. and the air bubbling is replaced with 98% oxygen bubbling. GC analysis after 36 hr total time indicates only 1.5% of compound 4 remains.

The reaction mixture is concentrated to a residue under vacuum and diluted with DCM (20 mL). The resulting slurry is filtered to remove NHPI. The filtrate is concentrated to ˜15 mL and solvent is swapped with MeOH using vacuum distillation. The mixture is diluted with MeOH (25 mL), cooled to 5-10° C. and filtered. The filter cake is washed with cold MeOH (5 mL) and dried under vacuum at 40-45° C. to afford 7.9 g of compound 5 as a light-green solid.

### Hydrolysis of Compound 5 to Compound 6:

To a solution of compound 5 (5 g) in DCM (75 mL) at 10-15° C. is added 32% conc. HCl (25 mL). The mixture is allowed to warm to 25-30° C. and held for 1.5 hr. Then the reaction mixture is diluted with water (50 mL) and the phases are separated. The aqueous layer is extracted with DCM (25 mL) and the combined DCM phases are washed with water (25 mL). The DCM is treated with activated carbon (0.25 g), held for 0.25 hr and filtered over filter-aid. The filter-aid cake is washed with DCM (15 mL) and concentrated to 5-10 mL under vacuum. The residue is diluted with n-heptane (25 mL) and concentrated again to 5-10 mL. The resulting mixture is diluted with n-heptane (25 mL), cooled to 5-10° C., held for 0.5 hr and filtered. The filter cake is washed with cold n-heptane (2.5 mL) and dried under vacuum at 40-45° C. to afford 3.8 g of compound 6 as a light-orange solid.

### Hydrogenation of Compound 6 to Compound 7:

Compound 6 (180 g), dichloromethane (DCM; 45 mL) and 3-picoline (1035 mL) were combined in a 2-liter autoclave. Diazabicyclo[2.2.2]octane (DABCO; 50.4 g) and 20% Pd(OH)₂ (50% water-wet, 7.2 g) were added. The resulting mixture was stirred at 26° C. under hydrogen gas at 6 bar pressure for 22 hr. The catalyst was then removed by filtration. The solid catalyst was washed with DCM (720 mL) and the filtrate was concentrated under vacuum to remove DCM. Water (1000 mL) was added and the mixture was concentrated under vacuum at 60° C. until the total volume was ˜360 mL. Toluene (1300 mL) was added the resulting solution was washed twice with 3N HCl (2×630 mL). The aqueous washes are combined and extracted with toluene (500 mL).

The toluene fractions were combined and washed with 3N HCl (255 mL) and then distilled under vacuum to ˜360 mL. 10% Aqueous ethanol (900 mL) was added and the solution was concentrated under vacuum to ˜360 mL. Additional 10% aqueous ethanol (900 mL) was added and again the mixture was concentrated under vacuum to ˜360 mL. Additional 10% aqueous ethanol (680 mL) was added and the mixture was cooled to 0-5° C. The slurry was filtered and the cake was washed with chilled (5-10° C.) 10% aqueous ethanol (85 mL). The cake was then dried under vacuum at 40-45° C. to provide 132.4 g (73.2% yield) of compound 7 as an off-white solid.

The solid was combined with additional lots of compound 7 to give 257 g. This was dissolved in DCM (514 mL) and 10% aqueous ethanol (1030 mL) was added. The resulting mixture was distilled under vacuum to a volume of ˜500 mL, and then additional 10% aqueous ethanol (1030 mL) was added. After concentrating under vacuum again to ˜500 mL, additional 10% aqueous ethanol (1030 mL) was added. The mixture was cooled to 0-5° C. and filtered, and the cake was washed with chilled (5-10° C.) 10% aqueous ethanol (125 mL). The cake was then dried under vacuum at 40-45° C. to provide 238 g (92.6% recovery) of compound 7 as an off-white solid.

Melting point=167° C.; Purity by CAD HPLC (w/w %)=98.7% (5α-impurity=0.37%); ¹H-NMR (400 MHz, CDCl₃): δ 3.67 (s, 3H), 2.90 (dd, J=12.8 & 6.5 Hz, 1H), 2.49 (t, J=11.4 Hz, 1H), 0.95-2.40 (m, 27H), 0.93 (d, J=6.4 Hz, 3H), 0.69 (s, 3H).

### Hydrolysis of Compound 7 to 3,7-DKCA:

To a solution of compound 7 (6 g) in IPA (30 mL/g) is added a solution of NaOH (1.5 g, 2.5 equiv) in water (30 mL) at room temperature. The reaction is warmed to 55-60° C. until it is found to be complete by TLC analysis.

The reaction mixture is concentrated to ˜30 mL to remove residual IPA and the resulting aqueous solution is washed with MTBE (2×30 mL). The aqueous phase is acidified to pH 2 using 6 M HCl, leading to the formation of a slurry. After cooling to 10-15° C., the slurry is filtered, washed with water and dried under vacuum at 45-50° C. to afford 4.2 g of 3,7-DKCA as a light-brown solid.

### Example 3: Preparation of UDCA from 3,7-DKCA

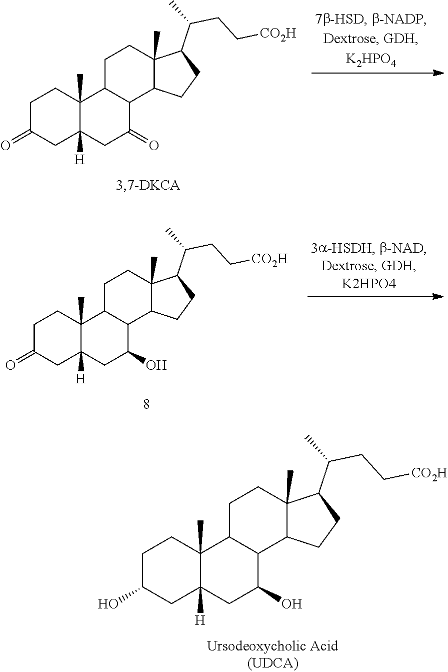

### Selective Reduction of the 7-Ketone of 3,7-DKCA to Provide Compound 8:

To a 250 mL single neck round bottom flask were added 3,7-DKCA (1 g, 2.57 mmol), dextrose (1.3 g), β-NADP (33 mg) and 250 mM K₂HPO₄ buffer (70 mL) at room temperature. The mixture was stirred for 0.5 h to get a clear solution. 7β-HSDH (66 mg) and GDH (2 mg) were added and the resulting mixture was stirred for 4 hr at room temperature. TLC analysis showed complete conversion of starting material.

The reaction mixture was quenched with 2N HCl solution until pH 3-3.5 was observed. The product was extracted in EtOAc (3×50 mL) and concentrated under reduced pressure to obtain compound 8 (1 g, as an off-white solid). ¹H NMR (400 MHz, MeOD): δ 3.49-3.58 (m, 1H), 2.70 (t, J=13.8 Hz, 1H), 2.48-2.29 (m, 3H), 2.28-2.15 (m, 2H), 2.14-2.05 (m, 5H), 1.98-1.78 (m, 7H), 1.72-0.95 (m, 15H), 0.71 (s, 3H)

### Selective Reduction of the 3-Ketone of Compound 8 to Provide UDCA:

To a 250 mL single neck round bottom flask were added compound 8 (1 g, 2.56 mmol), dextrose (1.3 g), β-NAD (33 mg) and 250 mM K₂HPO₄ buffer (70 mL) at room temperature. The mixture was stirred for 0.5 h to get a clear solution. 3α-HSDH (66 mg) and GDH (2 mg) were added and the resulting mixture was stirred for 20 hr at room temperature. TLC analysis showed complete conversion of starting material.

The reaction mixture was quenched with 2N HCl solution until the pH reached 3-3.5, and then the product was extracted in EtOAc (3×50 mL) and concentrated under reduced pressure to obtain UDCA (900 mg, as a white solid). ¹H NMR (500 MHz, MeOD): δ 3.44-3.53 (m, 2H), 2.30-2.38 (m, 1H), 2.18-2.25 (m, 1H), 2.03 (dt, J=6.5 Hz and 2.9 Hz, 1H), 1.95-1.79 (m, 5H), 1.56-0.92 (m, 26H), 0.71 (s, 3H)

### Example 4: Synthesis of TUDCA from UDCA

UDCA (5 g, 12.736 mmol) was charged to a 100 mL single neck round bottom flask. Acetone (30 mL, 6 vol) was added, resulting in a solution. Triethylamine (TEA, 1.7 mL, 0.97 equiv.) was added and the solution was cooled to 0° C. Ethyl chloroformate (1.34 g, 0.97 equiv.) was added and the resulting mixture was stirred for 4 h at room temperature under N₂ atmosphere. The reaction mixture was filtered to remove triethylamine hydrochloride and the filtrate was added dropwise to an aqueous solution of taurine sodium salt (prepared by reacting 1.9 g taurine with 0.6 g NaOH in 3.7 mL water) at room temperature over a period of 20 minutes. The reaction was continued for another 1 h at room temperature, at which point TLC analysis showed complete conversion.

Conc. HCl (1.5 mL), was added at room temperature to adjust the pH to ˜1. After stirring for 1 h, the resulting solid was filtered (0.9 g). The filtrate was diluted with acetone, stirred for 36 h at room temperature and the resulting solid was filtered, washed with acetone (10 mL) and dried under vacuum to obtain TUDCA as a white solid (5.1 g, 80% yield). ¹H NMR (400 MHz, CD₃OD): δ 3.62 (t, J=6.8 Hz, 2H), 3.52-3.42 (m, 2H), 2.97 (t, J=6.8 Hz, 2H), 2.38-2.28 (m, 1H), 2.20-2.10 (m, 1H), 2.08-2.00 (m, 1H), 1.92-1.75 (m, 5H), 1.65-0.96 (m, 21H), 0.95 (s, 3H), 0.70 (s, 3H); ¹³C NMR (100 MHz, CD₃OD): δ 177.97, 72.12, 71.96, 47.45, 56.34, 50.78, 44.79, 44.46, 43.99, 41.52, 40.69, 38.56, 37.96, 37.47, 36.89, 36.07, 35.15, 33.34, 33.18, 30.99, 29.63, 27.91, 23.93, 22.37, 18.96, 12.64; ESIMS for C₂₄H₄₀O₄ m/z 499.1 [M−H]⁺.

### Example 5: Preparation of TUDCA from 3,7-DKCA

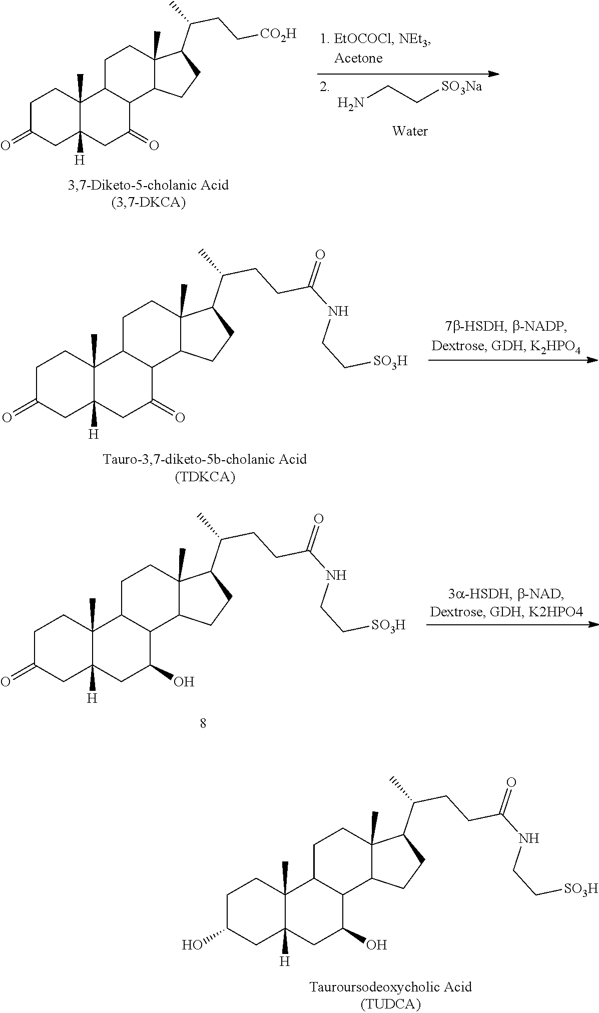

### Conversion of 3,7-DKCA to TDKCA:

To a solution of 3,7-DKCA (5 g) and triethylamine (1.75 mL, 0.97 equiv) in acetone (30 mL) at 0-5° C. is added ethyl chloroformate (1.2 mL, 0.97 equiv). The mixture is warmed to room temperature and held at this temperature until it is determined by TLC to be complete. The reaction mixture is filtered and the resulting filtrate is added dropwise to a mixture of taurine (1.93 g, 1.2 eq) and NaOH (0.62 g, 1.2 eq) in water (3.5 mL) at room temperature. The reaction mixture is held at this temperature until it is determined by TLC to be complete.

Conc. HCl (˜1.5 mL) is added to the reaction mixture until the pH is ˜1. The mixture is held for 1 h at room temperature and filtered. The filtrate is diluted with acetone (75 mL) and the resulting slurry is held for ½ hr at room temperature and filtered. The solids are washed with acetone (10 mL) and dried under vacuum to afford 4.5 g of TUDCA as a white solid.

### Selective Reduction of the 7-Ketone of TDKCA to Compound 8:

To a 100 mL single neck round bottom flask was added TDKCA (1 g, 2.02 mmol), dextrose (1 g) and β-NADP (33 mg) in 250 mM K₂HPO₄ buffer (65 mL) at room temperature. The mixture was stirred for 0.5 h to get a clear solution. To this solution 7β-HSDH (66 mg) and GDH (3.3 mg) were added and the reaction mixture was stirred for 18 h at room temperature and TLC analysis showed complete conversion of starting material.

The mixture was acidified using 6N HCl to pH˜1 and stirred for 1 hr. The product was extracted with n-BuOH (2×25 mL). The organic layers were combined and concentrated under vacuum until ˜3 mL of solvent remained. The slurry was diluted with acetone (30 mL) and stirred for 14 hr. The resulting slurry was filtered, washed with acetone and dried under vacuum to obtain 0.58 g of compound 8 as an off-white solid.

### Selective Reduction of the 3-Ketone of Compound 8 to Provide TUDCA:

To a 250 mL single neck round bottom flask are added compound 8 (1 g, 2.01 mmol), dextrose (1.3 g), 3-NAD (33 mg) and 250 mM K₂HPO₄ buffer (70 mL) at room temperature. The mixture is stirred for 0.5 h to get a clear solution. 3α-HSDH (66 mg) and GDH (2 mg) are added and the resulting mixture is stirred for 20 hr at room temperature. TLC analysis is expected to show complete conversion of starting material.

The reaction mixture is quenched with 2N HCl solution until the pH reaches ˜1, and then the product is extracted with butanol (3×25 mL). The organic fractions are combined and concentrated to ˜3 mL. The resulting mixture is diluted with acetone (30 mL) and stirring is continued for 15 hr. The resulting slurry is filtered to obtain TUDCA as a white solid.

### Example 6. 5B-Stereoselective Reduction Methods

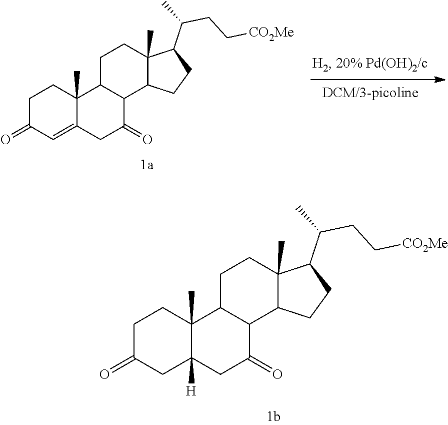

### Conversion of Compound 1a to Compound 1b:

A solution of compound 1a (690 g) in dichloromethane (DCM, 3100 mL), 3-picoline (1035 mL) and water (103 mL) was combined with 20% Pd(OH)₂-on-carbon (24.2 g, 50% water-wet) at 25° C. The reaction was warmed to 40° C., pressurized with hydrogen gas to 6 bar and held for 4 h with vigorous stirring. GC analysis indicated <0.1% of compound 1a remained.

The reaction mixture was filtered over Celite® and washed with DCM (3500 mL). The resulting filtrate was washed with 2 N HCl (6900 mL) and the organic phase was concentrated under vacuum to ˜1400 mL. The residue was diluted with isopropyl acetate (IPAc, 850 mL) and concentrated again to 1400 mL. The resulting mixture was diluted with IPAc (2000 mL), heated to dissolution at ˜60° C. and diluted with n-heptane (4000 mL) to afford a slurry. The slurry was cooled to 25° C. over 1 h, further cooled to 0° C. and held for 0.5 h. The solids were filtered and the filter cake was washed with a 1:2 mixture of IPAc/heptane pre-cooled to 5-10° C. (400 mL). The product was dried under vacuum at 40-45° C. to afford compound 1b as an off-white solid. The product was analyzed by GC and determined to contain 2% of the 5α-product.

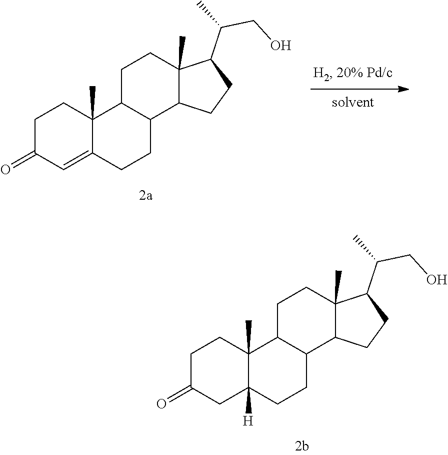

### Conversion of Compound 2a to Compound 2b (3-Picoline/Water Solvent):

A solution of compound 2a (1 g) in 3-picoline (11.4 mL) and water (0.6 mL) was combined with 20% Pd(OH)₂-on-carbon (50 mg, 50% water-wet) at 25° C. The reaction was warmed to 40° C., pressurized with hydrogen gas to 6 bar and held for 24 h with vigorous stirring. No compound 2a was detected by GC analysis.

A portion of the reaction mixture was treated with acetic anhydride and stirred for 30 mins at 25° C. The mixture was quenched with ice water, extracted into ethyl acetate. This ethyl acetate layer was washed with 1N HCl solution, concentrated and analyzed by GC. The analysis indicated that 92% of compound 2b was formed along with 3% of the 5α-product.

### Conversion of Compound 2a to Compound 2b (Isopropanol Solvent):

A solution of compound 2a (1 g) in isopropanol (12 mL) was combined with 20% Pd(OH)₂-on-carbon (50 mg, 50% water-wet) at 25° C. The reaction was warmed to 40° C., pressurized with hydrogen gas to 6 bar and held for 24 h with vigorous stirring. No compound 2a was detected by GC analysis.

A portion of the reaction mixture was treated with acetic anhydride and stirred for 30 mins at 25° C. The mixture was quenched with ice water, extracted into ethyl acetate. This ethyl acetate layer was washed with 1N HCl solution, concentrated and analyzed by GC. The analysis indicated that 55% of compound 2b was formed along with 33% of the 5α-product.

### Conversion of Compound 2a to Compound 2b (DCM/3-Picoline/Water Solvent):

A solution of compound 2a (2 g) in DCM, (18 mL), 3-picoline (6 mL) and water (1.2 mL) was combined with 20% Pd(OH)₂-on-carbon (50 mg, 50% water-wet) at 25° C. The reaction was warmed to 40° C., pressurized with hydrogen gas to 6 bar and held for 24 h with vigorous stirring. No compound 2a was detected by GC analysis.

A portion of the reaction mixture was treated with acetic anhydride and stirred for 30 mins at 25° C. The mixture was quenched with ice water, extracted into ethyl acetate. This ethyl acetate layer was washed with 1N HCl solution, concentrated and analyzed by GC. The analysis indicated that 94% of compound 2b was formed along with 5% of the 5α-product.

### Conversion of Compound 2a to Compound 2b (4-Picoline/Water Solvent):

A solution of compound 2a (1 g) in 4-picoline (6 mL) and water (0.3 mL) was combined with 20% Pd(OH)₂-on-carbon (50 mg, 50% water-wet) at 25° C. The reaction was warmed to 40° C., pressurized with hydrogen gas to 6 bar and held for 24 h with vigorous stirring. No compound 2a was detected by GC analysis.

A portion of the reaction mixture was treated with acetic anhydride and stirred for 30 mins at 25° C. The mixture was quenched with ice water, extracted into ethyl acetate. This ethyl acetate layer was washed with 1N HCl solution, concentrated and analyzed by GC. The analysis indicated that 96% of compound 2b was formed along with 2% of the 5α-product and 1% of the reductive amination product.

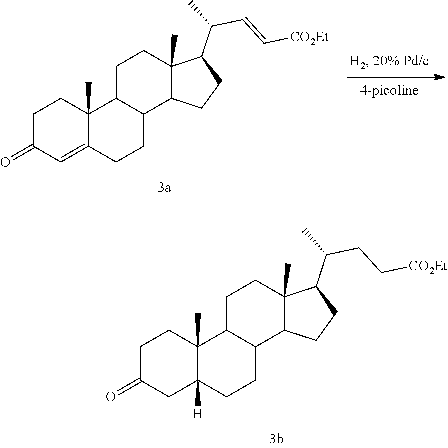

### Conversion of Compound 3a to Compound 3b (4-Picoline Solvent):

A solution of compound 3a (1.0 kg) in 4-picoline (5 L) was combined with 20% Pd(OH)₂-on-carbon (200 g, 50% water-wet) at 25° C. The reaction was pressurized with hydrogen gas to 30 psi and held for 7 h with vigorous stirring. No compound 3a was detected by TLC analysis.

The mixture was filtered over Celite® and the cake was washed with 4-picoline (2 L). The resulting solution was distilled under vacuum until no more 4-picoline was removed. DCM (10 L) and water (5 L) were added and the layers were separated. The aqueous phase was extracted with additional DCM (5 L). The combined DCM fractions were washed three times with 3N aqueous HCl (3×5 L) and then the DCM was removed via vacuum distillation. The resulting residue was slurried in petroleum ether (1 L) and filtered. After drying, 910 g of compound 3b was obtained. GC analysis showed 95% compound 3b, 3% of the 5α-isomer and 2% of the reductive amination products with 4-methylpiperidine.

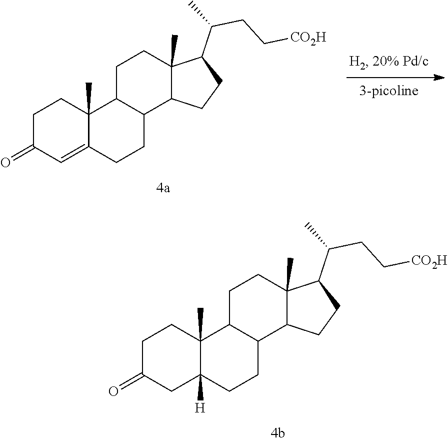

### Conversion of Compound 4a to Compound 4b (3-Picoline Solvent):

A solution of compound 4a (1.0 g) in 3-picoline (10 mL) was combined with 20% Pd(OH)₂-on-carbon (100 mg, 50% water-wet) at 25° C. The reaction was pressurized with hydrogen gas to 6 bar and held for 2 h with vigorous stirring. No compound 4a was detected by TLC analysis.

The mixture was filtered over Celite® and the cake was washed with 3-picoline (5 mL). The resulting solution was distilled under vacuum until no more 3-picoline was removed. The gummy residue was dissolved with DCM (10 mL) and the resulting solution was washed successively with 2 N HCl (10 mL) and water (10 mL). The DCM was removed under vacuum to provide 850 mg of compound 4b as a light-yellow solid.

A portion was converted to its ethyl ester and analyzed by GC to show 97% of compound 4b along with 3% of the 5α-isomer.

General Procedure for Conversion of Compounds 5a-9a to Compounds 4b-8b:

A solution of compound 5a-9a (1.0 g) in 3-picoline (10 mL) is combined with 20% Pd(OH)₂-on-carbon (100 mg, 50% water-wet) at 25° C. The reaction is pressurized with hydrogen gas (6 bar) and stirred vigorously until the reaction is confirmed by TLC to contain no more starting material.

The mixture is filtered over Celite® and the cake is washed with 3-picoline (5 mL). The resulting solution is distilled under vacuum until no more 3-picoline is removed. The residue is dissolved with DCM (10 mL) and the resulting solution is washed successively with 2 N HCl (10 mL) and water (10 mL). The DCM is removed under vacuum to provide the corresponding product. The product is analyzed by GC to provide product containing 95-99% of the 5β-product along with 1-5% of the 5α-product.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of reducing a 4,5-double bond on 3-ketochol-4-enoic acid (KCEA) or a derivative thereof defined by Formula I, to preferentially give a 5f-product, comprising contacting the compound of Formula I: 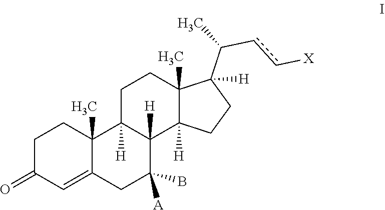
with a Pd catalyst in a solvent or solvent mixture comprising at least 10% of pyridine or a substituted pyridine, thereby producing the compound of Formula II: 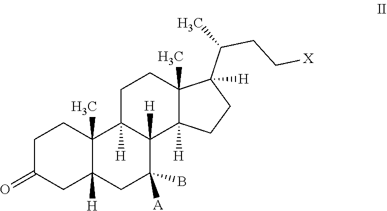
wherein:
a) A and B are OH and H respectively, H and OH respectively, H and H, or 7-oxo in combination;
b) X is C(O)OR¹ or C(O)NR¹R²; and
c) R¹ and R² are independently hydrogen, a counterion when the compound is a carboxylate or amide salt, optionally substituted C₁₋₂₀ alkyl, or optionally substituted aryl.

2) The method of claim 1, wherein the Pd catalyst is a heterogeneous catalyst.

3) The method of claim 1, wherein the Pd catalyst is Pd on carbon.

4) The method of claim 1, wherein the solvent comprises at least 10% of 3-picoline or 4-picoline or a combination thereof.

5) The method of claim 1, wherein the solvent comprises an organic cosolvent in combination with from 10% to 90% of the pyridine or substituted pyridine.

6) The method of claim 1, wherein the solvent comprises an organic cosolvent in combination with from 10% to 90% of 3-picoline or 4-picoline or a combination thereof.

7) The method of claim 1, wherein the solvent comprises an organic cosolvent in combination with from 20% to 40% of the pyridine or substituted pyridine.

8) The method of claim 1, wherein the solvent comprises an organic cosolvent in combination with from 20% to 40% of 3-picoline or 4-picoline or a combination thereof.

9) The method of claim 1 wherein the solvent further comprises from 1% to 20% water.

10) The method of claim 1 wherein the solvent further comprises from 2% to 5% of water.

11) The method of claims 1-10, wherein A and B are 7-oxo in combination.

12) The method of claims 1-10, wherein A and B are 7-oxo in combination and X is C(O)OR¹.

13) The method of claim 1, wherein A and B are H.

14) The method of claim 1, wherein A and B are H and X is C(O)OR¹.

15) The method of claim 1, wherein [symbol] is a single bond.

16) The method of claim 1, wherein [symbol] is a double bond.

17) The method of claim 1, wherein the compound of Formula I is selected from a compound of Formula 1a, 3a, 4a, 5a, 6a, 7a, or 9a, and the compound of Formula II is selected from a compound of Formula 1b, 3b, 4b, 5b, 6b, 7b, or 4b, respectively:

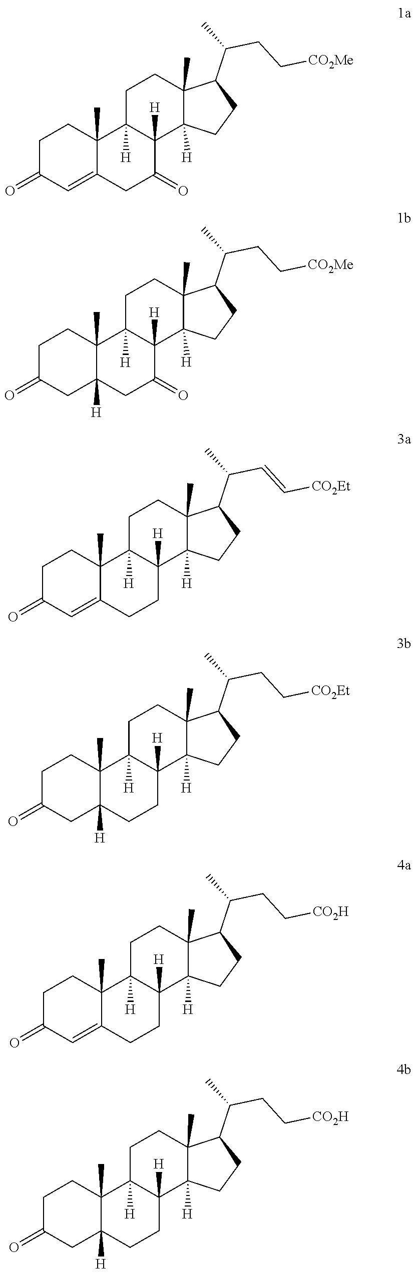

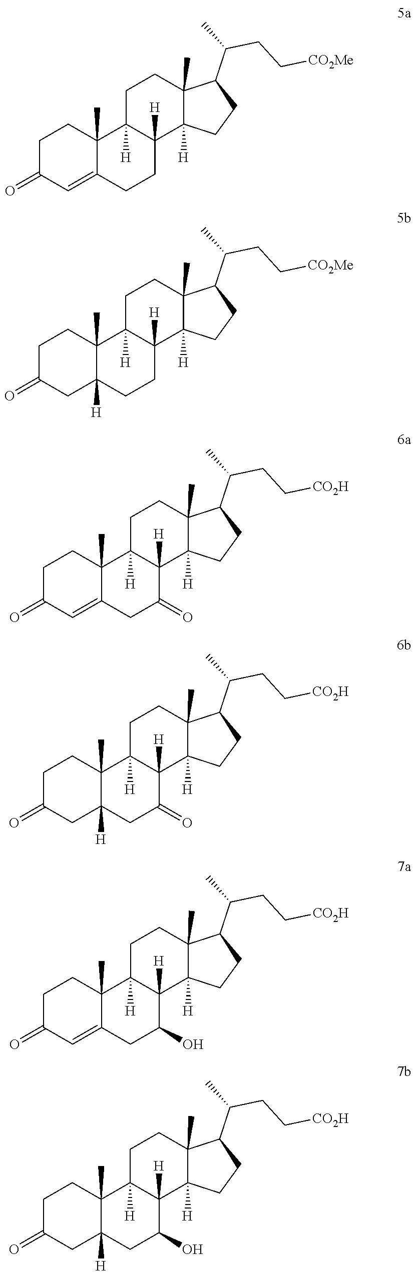

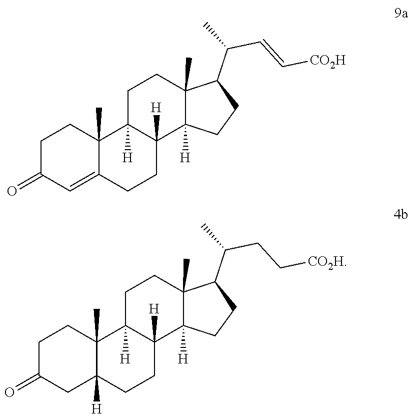

18) The method of claim 1, wherein A and B are 7-oxo in combination, further comprising:
a) when X is a C(O)OR¹ ester, hydrolyzing the ester;
b) when X is a C(O)NR¹R² amide, hydrolyzing the amide to C(O)OH;
c) reducing the 3-oxo to 3α-hydroxy, and
d) reducing the 7-oxo to 7β-hydroxy, to produce UDCA.

19) The method of claim 18, further comprising activating the carboxyl group of UDCA and reacting with taurine to produce TUDCA.

20) The method of claim 1, wherein A and B are H, further comprising:
a) when X is a C(O)OR¹ ester, hydrolyzing the ester;
b) when X is a C(O)NR¹R² amide, hydrolyzing the amide to C(O)OH;
c) reducing the 3-oxo to 3α-hydroxy, and
d) hydroxylating the 7-H to 7β-hydroxy, to produce UDCA.

21) The method of claim 20, further comprising activating the carboxyl group of UDCA and reacting with taurine to produce TUDCA.

22) The method of claim 1, wherein A and B are OH and H, respectively, further comprising:
a) when X is a C(O)OR¹ ester, hydrolyzing the ester;
b) when X is a C(O)NR¹R² amide, hydrolyzing the amide to C(O)OH; and
c) reducing the 3-oxo to 3α-hydroxy.

23) The method of claim 22, further comprising activating the carboxyl group of UDCA and reacting with taurine to produce TUDCA.

24) A method of reducing a 4,5-double bond on (20S)-21-hydroxy-20-methylpregn-4-en-3-one (BA) or a derivative thereof defined by Formula III, to preferentially give a 5β-product, comprising contacting the compound of Formula III: 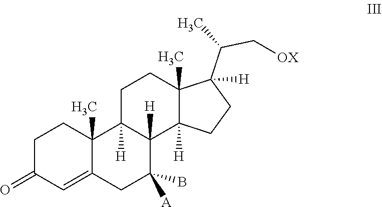
with a Pd catalyst in a solvent or solvent mixture comprising at least 10% pyridine or a substituted pyridine, thereby producing the compound of Formula IV: 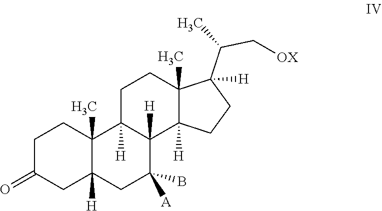
wherein:
a) A and B are OH and H respectively, H and OH respectively, H and H, or 7-oxo in combination; and
b) X is hydrogen or a protecting group.

25) The method of claim 24, wherein the Pd catalyst is a heterogeneous catalyst.

26) The method of claim 24, wherein the Pd catalyst is Pd on carbon.

27) The method of claim 24, wherein the solvent comprises at least 10% of 3-picoline or 4-picoline or a combination thereof.

28) The method of claim 24, wherein the solvent comprises an organic cosolvent in combination with from 10% to 90% of the pyridine or substituted pyridine.

29) The method of claim 24, wherein the solvent comprises an organic cosolvent in combination with from 10% to 90% of 3-picoline or 4-picoline or a combination thereof.

30) The method of claim 24, wherein the solvent comprises an organic cosolvent in combination with from 20% to 40% of the pyridine or substituted pyridine.

31) The method of claim 24, wherein the solvent comprises an organic cosolvent in combination with from 20% to 40% of 3-picoline or 4-picoline or a combination thereof.

32) The method of claim 24 wherein the solvent further comprises from 1% to 20% water.

33) The method of claim 24 wherein the solvent further comprises from 2% to 5% of water.

34) The method of claim 24, wherein X forms with the O to which it is attached an ester, an ether, a silyl ether, or an acetal.

35) The method of claim 24, wherein A and B are H.

36) The method of claim 24 wherein A and B are H and X forms with the O to which it is attached an ester, an ether, a silyl ether, or an acetal.

37) The method of claim 24, wherein A and B are H and X is H.

38) The method of claim 24, wherein A and B are OH and H, respectively.

39) The method of claim 24, wherein A and B are OH and H, respectively, and X forms with the O to which it is attached an ester, an ether, a silyl ether, or an acetal.

40) The method of claim 24, wherein A and B are OH and H, respectively, and X is H.

41) The method of claim 24, wherein the compound of Formula III is selected from a compound of Formula 2a or 8a and the compound of Formula IV is selected from a compound of Formula 2b or 8b, respectively:

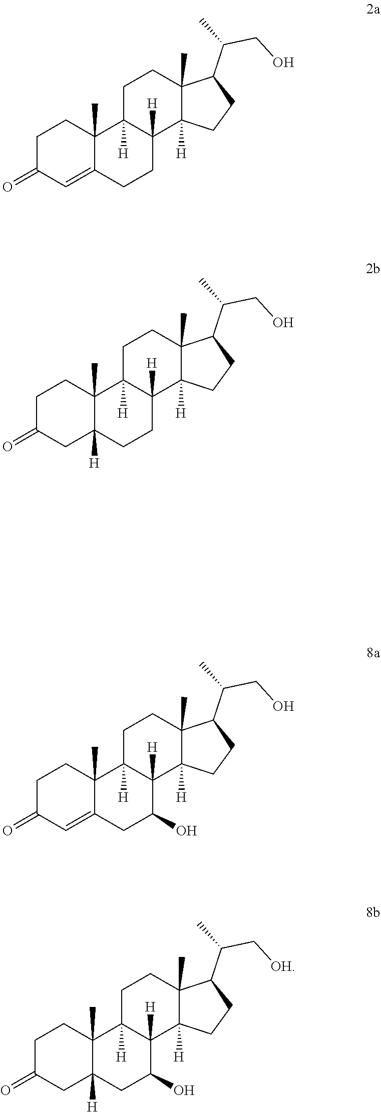

42) The method of claim 24, wherein A and B are H, further comprising:
a) converting the 21-alcohol group to a leaving group;
b) displacing the 21-leaving group with dialkylmalonate under basic conditions;
c) hydrolysis of both esters of the malonate group to give the dicarboxylic acid;
d) decarboxylation of the diacid to give the monoacid;
e) reducing the 3-oxo to 3α-hydroxy;
f) hydroxylating the 7-H to 7β-hydroxy, to produce UDCA; and
g) optionally activating the carboxyl group of UDCA and reacting with taurine to produce TUDCA.

43) The method of claim 24, wherein A and B are OH and H, respectively, further comprising:
a) selectively converting the 21-alcohol group to a leaving group;
b) displacing the 21-leaving group with dialkylmalonate under basic conditions;
c) hydrolysis of both esters of the malonate group to give the dicarboxylic acid;
d) decarboxylation of the diacid to give the monoacid;
e) reducing the 3-oxo to 3α-hydroxy, to produce UDCA; and
f) optionally activating the carboxyl group of UDCA and reacting with taurine to produce TUDCA.

44) A compound of Formula IV: 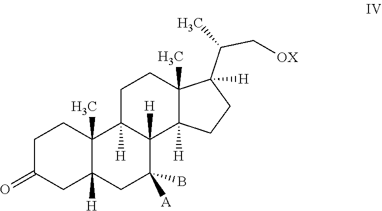
wherein:
a) A is OX;
b) B is H; and
c) each X independently forms OH or a protected OH, such as an ester, an ether, a silyl ether or an acetal,
or a salt thereof.

45) The compound of claim 44 which is (5β,7β,20S)-7,21-dihydroxy-20-methyl-pregnan-3-one.
